(19) United States
(12) Patent Application Publication
(10) Pub. No.: US 2014/0031726 A1
(43) Pub. Date: Jan. 30, 2014

(54) Low Frequency Ultrasound Surgical Systems for the Treatment of Soft Tissues

(71) Applicant: ENTrigue Surgical, Inc., San Antonio, TX (US)

(72) Inventors: Ary Chernomorsky, Walnut Creek, CA (US); Klimentina I. Nesterova, Omsk City (RU); Ivan Nesterov, Moskovskaya Obl (RU); Michael Rontal, Farmington Hills, MI (US); Peter Hwang, Stanford, CA (US)

(73) Assignee: ENTrigue Surgical, Inc., San Antonio, TX (US)

(21) Appl. No.: 13/941,256

(22) Filed: Jul. 12, 2013

Related U.S. Application Data
(60) Provisional application No. 61/671,181, filed on Jul. 13, 2012.

(51) Int. Cl. A61N 7/00

(52) U.S. Cl. CPC A61N 7/00
USPC 601/2

(57) ABSTRACT

Devices, systems and methods using low frequency ultrasound to emulsify, disintegrate, cut and/or coagulate tissue in a narrow space such as the nasal passages and paranasal sinuses. The devices may comprise a low frequency generator, a functionally active handle, an ultrasound emitter, a concentrator, a waveguide and distal functional tips. The devices and methods are configured to safely and efficiently carry out surgical procedures within the nose and sinuses, among other soft tissues within the body.

### CROSS-REFERENCE TO RELATED APPLICATIONS

This application claims priority to U.S. Provisional Application No. 61/671,181 filed Jul. 13, 2012, the entire contents which is specifically incorporated by reference herein without disclaimer.

### BACKGROUND

The bony nasal passage is narrow with a 1 to 5 mm width between the midline nasal septum and the turbinates of the lateral wall. The lateral nasal wall is a complicated and convoluted series of projecting bony structures called turbinates, and the sinuses are lateral thereto. Covering these bony structures is a very vascular soft tissue. The total distance from septum to orbit is only 12-15 mm anteriorly and 12-25 mm posteriorly.

Surgery into the nose through the nostril has developed as new endoscopes have allowed visualization and sophisticated hand tools have been devised. The latter allows one hand to dissect while the other holds and directs the endoscope. As small as these are, there is competition for room to work between the endoscopic equipment and even the most up-to-date of instruments.

Diseases of the nose are often manifestations of the very vascular and complicated nature of the nasal surfaces. The perversions of the normal functioning of the nasal mucosa to warm, humidify, clean and test the inhaled air leads to pathologic tissues, bacterial accumulations and inflammatory reactions on and within the mucosa. There are masses such as polyps, projections of cartilage and bone from abnormal growth and nasal trauma, scarring from disease and iatrogenic events, swelling and hypertrophy of vascular tissue within the mucosa and idiopathic bleeding. All of these decrease the space within the nose and lead to obstruction, infection, discharge and facial pain.

Conventionally, the surgical treatment of these nasal and sinus conditions is performed with small cold steel-powered and unpowered instruments guided by endoscopic viewing equipment and the addition of various radiofrequency (RF) coagulation devices. Using such instruments, polyps can be removed, scars can be taken down, hypertrophied mucosa overlying turbinates can be reduced, cartilage and bone can be removed from the septum for airway improvement and bleeding can be coagulated.

The turbinates are reduced in size by placing an RF needle into the swollen mucosa and destroying the submucosa and at times the mucosa. Alternatively, a cold technique can be used, whereby an incision in made into the turbinate mucosa, the soft tissue is elevated off of the bone and this soft tissue is cut and sucked out, while endeavoring to leave the overlying mucosa uninjured. Reduction of these inferior turbinates allows significant relief of nasal obstruction. Problems are associated with each of these techniques such as, for example, the inability to control intra-turbinate coagulation and even the coagulation and eventual scarring of the overlying mucosa. One problem with the cold technique is related to the profuse bleeding that can occur from within the gutted turbinate, which conventionally requires this procedure to be carried in an operating room setting.

Scars are conventionally removed by cutting with cold or RF devices and held apart until the mucosa heals. In either case, the resulting raw surfaces must be held apart until the mucosa can grow across the exposed underlying submucosal tissue. This requires the placement of packing material in the nasal cavity for a period of days. Moreover, the poor control over the extent of destruction of tissues using the RF device often results in re-scarring that may re-obstruct the passages sought to be re-opened by the procedure.

Projections from the septum, which consist of cartilage and bone, are conventionally removed by elevating the mucosa from these in a cold fashion removing the cartilage and bone and fastening the mucosa down afterward. This necessitates the creation of a significant muco-perichondrial or muco-periosteal flap. The bone or cartilage is removed and the flap created must be held down to prevent bleeding and perforation. This is accomplished with either packs or some suturing technique. One drawback of this approach is that the relatively large flap that must be elevated for the small amount of underlying cartilage or bone, as well as the difficulty in removing bone and cartilage in a controlled fashion and the need for bulky and uncomfortable packing afterward. Septal deformation is a possibility.

Polyps are conventionally removed by grasping and avulsing them with grasping forceps or by piecemeal removal with powered cutting shavers. These polyps can be quite vascular, especially at their base. Intra-operative removal must be as bloodless as possible to allow continuous visual control and to enable the procedure to proceed in an uninterrupted fashion, without having to constantly stop to clear blood from the field and endoscope. Today, intra-operative control of the bleeding depends on pre-resection vasoconstriction. At the end of surgery, bloody ooze is conventionally controlled with packing or chemical coagulation suspensions. In either case, this is uncomfortable to the patient as the nose is obstructed.

Bleeding in the nose is conventionally controlled by cautery, either chemical or by RF devices or by pressure packing In the face of patient anticoagulation, the problem can be serious with significant amounts of blood lost until the source is controlled. Posterior packing that traps the blood between the anterior and posterior openings of the nose can result in significant blood pressure and oxygenation problems. In addition, electrocautery destruction of mucosa and the underlying blood vessels leaves a deep ulcer into the submucosal tissue that heals slowly. At times, this does not heal and a through and through perforation is produced. In practice, these ulcers and perforations are sources for later bleeding as the crusts lift off and often lead to re-bleeding.

The problems encountered with these procedures vary. However, these procedures share some common problematic issues; namely, the control of effect and bleeding. The procedures need to be carried out in the narrow confines of the nose and paranasal spaces. It is worthy of note that these procedures are commonly considered to be one surgeon procedures that require one hand to hold an endoscope for visualization and the other hand to perform the procedure. In all of the above procedures, the hand wielding the active tool is attempting to achieve a desired therapeutic result while simultaneously controlling bleeding.

For polypectomy, the coagulated and emulsified submucosal tissue as well as the mucosa must be made bloodless and shrink so that the polyp can be removed. Conventional cutting instruments developed for polypectomy generate excess bleeding, which obscures the operative field. For lack of good clear visualization, too many times polyp removal takes the surgeon blindly to the outer reaches of the operative field ending at the skull base or orbital wall, with potentially negative outcomes for the patient.

As to bleeding, whether from nasal surgery or idiopathic epistaxis, the procedure needs to be quick and painless for office procedures and efficient in causing coagulation in the operating room. Painless means having to use minimal local anesthesia such as topical anesthetics or minimal injections into the mucosa as opposed to trying to cause major regions of anesthesia and vasoconstriction with nerve/vascular blocks.

Finally, nasal procedures today need to return to the office where they were done for so many years. The driving motivation for taking cases to the operating room was the need to control pain, control bleeding and have access tools that were otherwise too expensive or too dangerous for office use. Needed is a device that can reduce, cut and coagulate nasal tissue while causing little bleeding and requiring no packing Such a device would enable many of the aforementioned procedures to be carried out in an office setting.

### SUMMARY

The devices and methods shown and described herein use low frequency ultrasound to thermally ablate, disintegrate, emulsify, reduce, cut and/or coagulate tissue in a narrow space such as the nasal passages, paranasal sinuses and nasal pharynx employing specially designed generators, unique waveguides and functionally active handpieces. These devices and methods are configured to safely and efficiently carry out surgical procedures within the nose, nasal pharynx and sinuses, though those familiar with the art will readily recognize that other applications are possible. Advantageously, using the present embodiments, traditional OR procedures may be transformed into a single or a series of office procedures for greater efficiency and patient comfort. Also, certain conditions may be treated earlier than would otherwise be the case, due to the low risk associated with the use of the present embodiments in an office setting. Accordingly, one embodiment is a device that may comprise an ultrasonic emitter, the ultrasound emitter being configured to be coupled to an ultrasound generator; a handle, the handle comprising an external surface and an internal surface that defines an interior cavity configured to at least partially encapsulate the ultrasonic emitter; a compressible (e.g., soft) material disposed within the internal cavity, the compressible material being configured to support the ultrasonic emitter within the internal cavity; and a waveguide assembly, the waveguide assembly comprising a proximal end coupled to the ultrasonic emitter and a distal end comprising a distal functional tip.

According to further embodiments, the compressible material may comprise acoustically isolating material. The compressible material may comprise one or more annular supports configured to surround the ultrasound emitter within the internal cavity of the handle. The compressible material may comprise a ball defining a through bore through which the ultrasound emitter may be disposed. The compressible material may be configured to isolate the ultrasound emitter from the internal surface of the handle. The handle may comprise a first half and a second half, the first and second halves are configured to couple to one another. The device may further comprise a hermetic collar configured to provide a fluid-tight seal between the handle and at least a portion of the waveguide assembly. The device may further comprise a movement generating mechanism configured to cause a repetitive movement of the distal functional tip of the waveguide assembly. For example, the movement generating mechanism may comprise an electric motor assembly. The electric motor assembly may be coupled to the compressible material supporting the ultrasonic emitter within the internal cavity. For example, the electric motor assembly may comprise an eccentric motor. The handle generally may define a pistol grip shape, for example. The device may further comprise a trigger coupled to the handle, the trigger being configured to cause the ultrasound emitter to move within the handle when depressed. At least the exterior surface of the handle may comprise a porous layer.

The waveguide assembly may comprise a concentrator, a waveguide and a distal functional tip. The waveguide assembly may comprise a detachable concentrator detachably coupled to the ultrasonic emitter. The concentrator may define a conical shape or a surface having a shape of a Gaussian curve, for instance. The waveguide assembly may comprise a detachable cylindrical waveguide. The waveguide assembly may comprise a detachable distal functional tip. The waveguide assembly may comprise a disposable distal functional tip configured for a predetermined number of uses (a single or a finite greater number of uses, for example). The waveguide assembly may comprise a distal functional tip that is associated with and/or configured to store a unique identifier. The distal functional tip may comprise a communication device that configured to be polled, to enable a running count of uses of the distal functional tip to be maintained. The ultrasound emitter may comprise a communication unit configured to communicate at least with the communication unit of the distal functional tip.

According to one embodiment, an ultrasound soft tissue management system may comprise an ultrasound generator; an ultrasound emitter configured to couple to the ultrasound generator; a handle configured to at least partially encapsulate the ultrasound emitter; and a waveguide assembly, the waveguide assembly comprising a proximal end coupled to the ultrasonic emitter and a distal end comprising a distal functional tip configured to store a unique identifier.

The waveguide assembly may be configured to enable the distal functional tip to be detachable. The distal functional tip may comprise a communication device that may be configured to provide the unique identifier when polled. The ultrasound emitter and/or the ultrasound generator may be configured to poll the waveguide assembly to obtain the unique identifier of the distal functional tip. The ultrasound emitter and/or the ultrasound generator may be configured to maintain a running count of uses of the distal functional tip using the obtained unique identifier. The ultrasound emitter and/or the ultrasound generator may be configured to disallow further uses of the distal functional tip after the running count has reached a predetermined threshold (such as one or a predetermined finite number greater than one). The ultrasound generator may comprise a non-volatile memory configured to store the running count of uses of the distal functional tip. The unique identifier may be associated with information identifying the distal functional tip. The ultrasound generator may be configured to reconfigure signals applied to the ultrasound emitter, based on the identifying information associated with the obtained unique identifier of the distal functional tip. The ultrasound generator may be configured to reconfigure the frequency, power, amplitude and/or on/off duty cycle of signals to be applied to the ultrasound emitter, based on the identifying information associated with the obtained unique identifier of the distal functional tip. The unique identifier may be associated with information identifying a procedure to be carried out using the distal functional tip.

One embodiment is an ultrasound soft tissue management system, comprising an ultrasound emitter configured to couple to an ultrasound generator; a handle configured to at least partially encapsulate the ultrasound emitter; and a waveguide assembly, the waveguide assembly comprising a proximal end coupled to the ultrasonic emitter and a distal end; and a distal functional tip; and a connecting tool configured to at least partially encapsulate the distal functional tip and to enable the distal functional tip to be coupled to the distal end of the waveguide assembly.

According to other embodiments, the connecting tool may comprise an internal surface defining a generally distal functional tip-shaped void therein. The connecting tool may be configured to couple the distal functional tip to the distal end of the cylindrical waveguide assembly with a predetermined amount of torque. The connecting tool may comprise a coupling feature configured to couple to a torque wrench. The connecting tool and at least partially encapsulated distal functional tip may be configured to be heated or cooled prior to affixation of the distal functional tip on the distal end of the waveguide assembly.

A method of operating an ultrasonic soft tissue treatment device, according to one embodiment, comprises detecting an affixation, to the ultrasonic soft tissue treatment device, of one of a plurality of distal functional tips configured to deliver ultrasound energy to soft tissue, each of the plurality of distal functional tips being configured to provide a unique identifier when polled; polling the detected distal functional tip to obtain the unique identifier thereof; configuring signals to be applied to the ultrasonic soft tissue treatment device based at least upon the obtained unique identifier; and energizing the ultrasonic soft tissue treatment device to cause the configured signals to be delivered to the ultrasonic soft tissue treatment device, transduced into ultrasound energy and delivered to the distal functional tip.

Configuring may comprise configuring the amplitude, frequency, power and/or on/off duty cycle of the signals to be applied to the ultrasound emitter assembly. The unique identifier may be associated with the type of the detected distal functional tip and/or with one or more procedures associated with the use of the detected type of distal functional tip. The method may further comprise inserting the distal functional tip into the soft tissue to apply ultrasonic energy thereto and energizing a motor that causes the distal functional tip to repetitively move within the soft tissue. The method may also comprise determining a number of uses that the detected distal functional tip has undergone, based upon the obtained unique identifier. Use of the detected distal functional tip may be enabled if the determined number of uses is less than a predetermined threshold and may be disallowed if the determined number of uses is greater or equal to the predetermined threshold.

According to one embodiment, a device may comprise an ultrasonic emitter, the ultrasound emitter being configured to be coupled to an ultrasound generator; a waveguide assembly, the waveguide assembly comprising a proximal end coupled to the ultrasonic emitter and a distal end comprising a distal functional tip, wherein the ultrasonic emitter and waveguide assembly are integrated into a single, non-detachable assembly; and a handle, the handle comprising an external surface and an internal surface that defines an interior cavity that is configured to at least partially encapsulate the ultrasonic emitter. The handle may comprise a first half and a second half, wherein the first and second halves are configured to couple to one another. The handle may further comprise a hermetic collar configured to provide a fluid-tight seal between the handle and at least a portion of the waveguide assembly. The device may further comprise a compressible material disposed within the internal cavity, the compressible material being configured to support the ultrasonic emitter within the internal cavity. The compressible material may comprise acoustically isolating material. The compressible material may comprise at least one annular support configured to surround the ultrasound emitter within the internal cavity of the handle. The compressible material may comprise a ball defining a through bore through which the ultrasound emitter is disposed. The compressible material may be configured to isolate the ultrasound emitter from the internal surface of the handle. The may further comprise a movement generating mechanism that is configured to cause a repetitive movement of the distal functional tip of the waveguide assembly. The movement generating mechanism may comprise an electric motor assembly, the electric motor assembly being coupled to the compressible material supporting the ultrasonic emitter within the internal cavity. The electric motor assembly may comprise an eccentric motor. The handle may generally define a pistol grip shape. The device may further comprise a trigger coupled to the handle, the trigger being configured to cause the ultrasound emitter to move within the handle when depressed. The exterior surface may comprise a porous layer. The waveguide assembly may comprise an integrated concentrator, waveguide and distal function tip. The concentrator may define a conical shape or a surface having a shape of a Gaussian curve.

### BRIEF DESCRIPTION OF THE DRAWINGS

FIG. 1 is a diagram of an ultrasound emitter, according to one embodiment.

FIG. 2 is a diagram of an ultrasound emitter coupled within a handle, according to one embodiment.

FIG. 3 is a diagram showing aspects a hinged handle, according to one embodiment.

FIG. 4 shows a cross section of the nasal turbinates and the distal functional tip of a low frequency ultrasound device inserted therein, according to one embodiment.

FIG. 5A is a diagram of an ultrasonic emitter coupled within a handle, according to one embodiment.

FIG. 5B is a diagram of an ultrasonic emitter coupled within a handle, according to one embodiment.

FIG. 6 is a diagram of an ultrasound emitter coupled within a handle, according to one embodiment.

FIG. 7 is a diagram of an ultrasonic emitter comprising a reciprocating mechanism, according to one embodiment.

FIG. 8 is a diagram of an ultrasonic emitter comprising offset visualization optics, according to one embodiment.

FIG. 9 is a diagram of an ultrasonic emitter configured for selective attachment of different cylindrical waveguides and distal functional tips, according to one embodiment.

FIG. 10 is a diagram of a handle of an ultrasonic emitter, according to one embodiment.

FIG. 11 is another diagram of a handle of an ultrasonic emitter, according to one embodiment.

FIG. 12 is a flowchart of a method of carrying out a procedure within a nasal cavity, according to one embodiment.

FIG. 13 shows a variety of detachable distal functional tips configured for use with the present ultrasound emitter, according to embodiments.

FIG. 14 shows methods and structures for attaching a detachable distal functional tip to a cylindrical waveguide of an ultrasonic emitter, according to embodiments.

FIG. 15 shows a soft tissue management system, according to embodiments.

FIG. 16 is a block diagram of a soft tissue management system, illustrating the selective modularity of the constituent components thereof, according to embodiments.

FIG. 17 is a method of operating an ultrasonic soft tissue treatment device, according to one embodiment.

### DETAILED DESCRIPTION

The devices and methods shown and described herein use low frequency ultrasound to disintegrate, emulsify, reduce, cut and/or coagulate tissue in a narrow space such as the nasal passages, paranasal sinuses and nasal pharynx employing specially designed generators, unique waveguides and functionally active handpieces. These devices and methods are configured to safely and efficiently carry out surgical procedures within the nose and sinuses, though those familiar with the art will readily recognize that other applications are possible.

One embodiment comprises an assembly comprising an ultrasonic powered generator, a handle and an integrated ultrasound emitter having a distal functional tip that is configured to enter tissue, disintegrate, cut and coagulate, in a substantially bloodless manner. The results are predictable, almost bloodless and leave the patient with an open post procedure nasal passage (no packing) and it is markedly less painful.

According to one embodiment, the present ultrasonic device with specially designed waveguides can burrow into a turbinate with little pressure and immediately begin coagulating the deep tissue between the turbinate bone and the overlying mucosa. The amount of energy needs to be predetermined both in terms of power and time of exposure to achieve the desired result. The same can be said for cutting scars where no tissue needs to be removed.

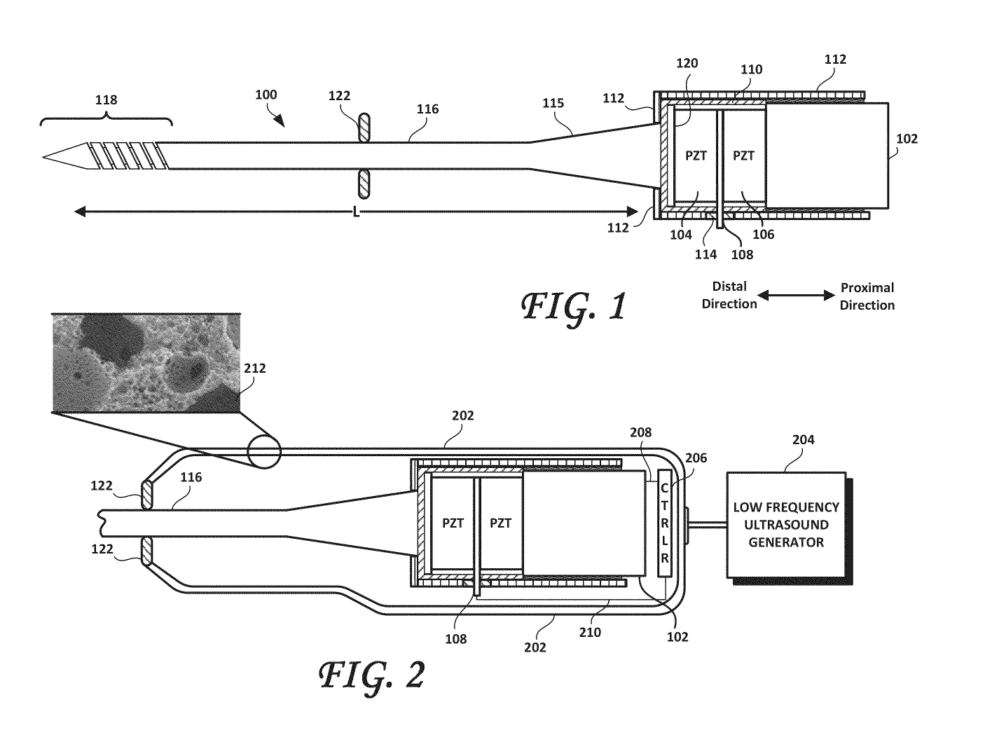

FIG. 1 is a diagram of an integrated ultrasound emitter 100, according to one embodiment. As shown therein, the ultrasound emitter 100 may comprise a proximal reflector 102 threaded or otherwise coupled to a housing 112. The proximal reflector 102 may comprise titanium, for example. The housing 112 may also enclose a pair of piezoelectric transducers 104, 106, separated by an electrode 108. Tightening of the threaded proximal reflector 102 within the housing 112 compresses the pair of piezoelectric transducers 104, 106 and the electrode 108 located there between to provide a tight mechanical and acoustic contact between the proximal reflector 102 and the hub flange 120 of a conical concentrator 115. When a suitable voltage is applied to the electrode 108 and to the proximal reflector 102 (for example), the piezoelectric transducers 104, 106 expand and contract to generate ultrasonic waves. Because of the large mass of the proximal reflector 102, the majority of the power of the generated ultrasonic waves is reflected in the distal direction, toward the concentrator portion of the waveguide 115, the cylindrical portion of the waveguide 116 toward the distal functional tip 118. As a result of the reduction in cross-section or density of the concentrator portion 115 to the cylindrical portion of the waveguide 116, the particle vibration displacement amplitude is increased. It is to be noted that the concentrator portion 115 of the waveguide may be conical in shape or may define a more complex geometry, such as a Gaussian curve, for example. The displacement amplitude increases with the difference in the diameters or densities of opposite ends of the integrated ultrasound emitter 100. As the integrated ultrasonic concentrator may be configured to operate at or near resonance, the length L of the integrated ultrasonic concentrator should be a multiple of the half-waves integer, such that L should be about nλ/2, where n=1, 2, 3.

The integrated ultrasonic emitter 100, according to one embodiment, may be configured for repeated use. Therefore, the internal components thereof should be both isolated from the environment and insulated, to prevent current from flowing from, for example, the proximal reflector 102 to the electrode 108, thereby creating a short circuit between these two points. The integrated ultrasonic emitter 100 may be configured, therefore, to undergo repeated sterilizations in an autoclave, for example.

Toward that end, one embodiment comprises a hermetic plug 114 disposed within an opening in the housing 112 through which the electrode 108 passes. The hermetic plug 114 may, for example, be formed of a silicone medical grade elastomer such as, for example, Silastic® MDX4-4210. Silicone is not overly heat sensitive, is not destroyed by vibrations and exhibits high elasticity. Moreover, one embodiment comprises an insulation layer 110 disposed between the housing 112 and the piezoelectric transducers 104, 106 to insulate and isolate the piezoelectric transducers 104, 106. The insulation layer 110 may be very thin, so as to avoid dampening the ultrasonic vibrations and/or may be isolated from vibrating parts of the emitter. Alternatively, in one embodiment, reference 110 in FIG. 1 may denote one or more layers of UV curable Medical Grade adhesives, such as SuperBonder, Hysol and Flashcure, made by Loctite®.

One embodiment of the ultrasound emitter 100 may also comprise a hermetic collar 122, configured to further isolate the body of the integrated ultrasound emitter from moisture, such as bodily fluids encountered during a procedure. The hermetic collar 122 may be coupled to the waveguide 116. However, it should be noted that coupling any significant rigid mass to the cylindrical waveguide 116 may result in unwanted dissipation of acoustic energy and therefore, potential loss or quenching of the resonance condition. However, the use a thin elastomeric “lip” or collar 122 comprising lightweight soft silicone material, such as MED-4805 LSR elastomer by NuSil Inc., may significantly minimize such energy loses.

FIG. 2 is a diagram of an ultrasound emitter coupled within a handle, according to one embodiment. As shown, a handle 202 may be coupled to the ultrasound emitter 100. The handle 202 may be reusable or configured for one-time use (i.e., disposable). The handle 202 may be configured to accommodate, enclose and seal the ultrasound emitter 100 from bodily fluids and the environment. According to one embodiment, the handle 202, for example, may comprise first and second halves that are hinged together so that the handle 202 may be opened, the ultrasound emitter 100 disposed therein and securely closed, so as to encapsulate the ultrasound emitter 100 within the internal cavity formed by the first and second halves of the handle 202.

The ultrasound device of FIG. 2 is shown coupled to a low frequency ultrasound generator 204. Specifically, the low frequency ultrasound generator 204 may be coupled to a controller/piezoelectric transducer driver 206, which may be disposed within the handle 202. The controller/piezoelectric transducer driver 206 may be electrically coupled to the ultrasound emitter 100 through an electrical connection 208 coupled to, for example, the proximal reflector 102 and through a second electrical connection 210 coupled to the electrode 108 disposed between the two piezoelectric transducers 104, 106. The piezoelectric transducers 104, 106, for example, may be configured to generate ultrasonic energy within a therapeutic frequency range. For example, the piezoelectric transducers 104, 106 may be configured for generating a specific frequency for a specific soft tissue therapeutic intervention. For example, the piezoelectric transducers 104, 106 may be configured to generate ultrasonic energy within a frequency range of about 20 kHz to about 40 kHz. For instance, the piezoelectric transducers 104, 106 may be configured to generate ultrasonic energy between 23 kHz and 28 kHz. For example, the piezoelectric transducers 104, 106 may be configured to generate ultrasonic energy at about 25 kHz. Spring contacts, for example, between the handle 202 and the electrical connections 208, 210 may be provided to ensure good electrical contacts with the proximal reflector 102 and the electrode 108. A switch coupled to the controller/piezoelectric transducer driver 206 may be mounted to the handle 202 to enable the operator to selectively turn the ultrasound device on and off.

The low frequency ultrasound generator 204 may be configured, for example, to output a peak-to-peak voltage from about 40V to about 160V. For example, the generator 204 may be configured to output a peak-to-peak voltage from about 60V to about 120V. For example, the generator 204 may be configured with an output power of about 25 watts with a peak-to-peak output voltage of about 80V, when tuned at resonance.

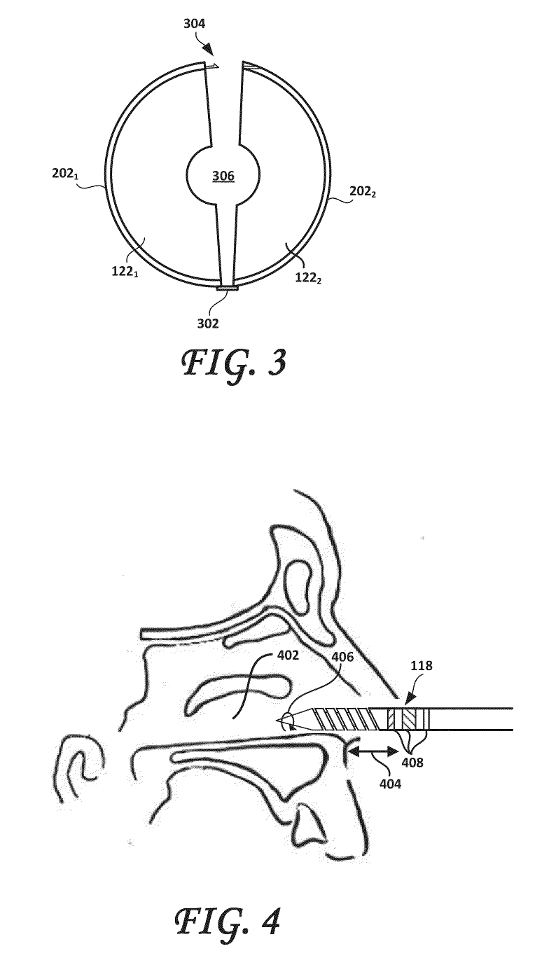

According to one embodiment, to further seal the ultrasound emitter 100 from bodily fluids and the environment, the hermetic collar 122 may provide a fluid-tight seal between the waveguide 116 and the handle 202. Toward that end, the hermetic collar 122 may be disposed at or near the distal end of the handle 202 as shown in FIG. 2 or may be disposed further in the proximal direction. The hermetic collar 122 may be coupled to the waveguide 116. According to one embodiment, the hermetic collar may be coupled (by an adhesive, for example) to the handle 202 such that when the ultrasound emitter 100 is placed with the first and second halves of the handle 202 and the two hinged halves closed, a hermetic seal is produced between the waveguide 116 and the handle 202. According to one embodiment shown in FIG. 3, the hermetic collar 122 may comprise first and second collar halves 122₁ and 122₂, each of the collar halves 122₁ and 122₂ being coupled to a respective one of the handle halves 202₁ and 202₂, as shown in FIG. 3. The handle halves 202₁ and 202₂ may be coupled to one another by a hinge mechanism 302, for example. Alternatively, reference 302 may be a relatively thin and bendable seam joining the two halves 202₁ and 202₂ of the handle 202. As the two halves 202₁ and 202₂ of the handle 202 are closed, the cavity 306 formed by the first and second collar halves 122₁ and 122₂ comes into intimate contact with the waveguide 116, thereby sealing the proximal portion thereof and the ultrasound emitter 100 from bodily fluids and the environment. A latch mechanism 304 may be configured to lock the first handle half 202₁ to the second handle half 202₂ when the handle 202 is closed to lock the ultrasound emitter 100 therein.

The ultrasound emitter 100, according to one embodiment, may be configured for the surgical treatment of various pathological ENT conditions. The ultrasound emitter 100 may be a single use device or may be configured to be re-usable and configured to be coupled with a variety of different waveguides, each of the waveguides 116 being provided with different distal functional tips 118. According to one embodiment, the handle 202 may be single use and disposable. Alternatively, according to one embodiment, the handle 202 may be configured for multiple uses and may be sterilized in an autoclave, for example.

Ultrasonic vibration generated in and by the emitter 100 are predominantly transmitted toward the distal functional tip 118. Nonetheless, there is a certain amount of vibration that is transmitted to the body of the handle 202 and thereafter to the operator's hand grasping the handle 202. After a long period of operation, such vibrations may become tiresome and may eventually impair the user's ability continue effective use of the assembly. Indeed, such vibrations may contribute to the development or exacerbation of pathological conditions such as Vibration Syndrome, Vibration-induced White Finger Syndrome (VWF) and even Carpal Tunnel Syndrome. According to one embodiment, the handle 202 may comprise vibration-absorbing material 212. The vibration-absorbing material 212 may be provided as an external layer on the handle 202. Alternatively, the handle 202 may be formed of or otherwise comprise vibration-absorbing material 212. For example, the handle 202 may be formed at least partially of or may comprise a layer of vibration absorbing material 212 such as, for example, Nu 202 composite vibration reducing gel by Ergodyne of St. Paul, Minn. According to one embodiment, the material (s) from which the handle 202 may be formed may comprise an internal porous layer defining a plurality of voids and/or air pockets. Such voids and/or air pockets (an example of which is shown at 212 in FIG. 2) may be formed during the process of manufacturing the handle 202. Such structure may be accomplished through injection of the air or other gases into the polymer material from which the handle 202 may be fabricated during, for example, an injection molding process. For example, a suitable material for inclusion in or manufacture of the handle 202 is Ultem® PolyEtherlmide (PEI) produced by Boedeker Plastics, Inc. Variations in induced porosity may create different anti-vibration configurations for the handle 202 and/or selected portions thereof. The porosity density and/or size and/or distribution and/or shape of the voids/air bubbles within the material may be freely selected by varying the manufacturing parameters of the material and injection molding process, thereby changing the vibration-absorbing characteristics of the resultant material.

Different waveguides and different configurations of the distal functional tip 118 may be used for different procedures. For example, nasal turbinate reduction is one of the most popular minor surgeries carried out by the ENT practitioner. A titanium waveguide and distal functional tip 118 called a disintegrator is one of the most powerful of waveguides, and is designed to thermally ablate the cavernous blood vessels of the nasal cavities. In operation of the low frequency ultrasound device according to one embodiment, the mechanism of thermal ablation may be triggered by cavitation, and may be a actually secondary mode of action. This mechanism thermally ablates tissue that is extremely rich in blood vessels. To carry out such thermal ablation, the distal functional tip 118 may be introduced within the nasal turbinates and thereafter energized. The ultrasonic energy generated at the distal functional tip causes the ablation of the highly vascularized turbinate tissues. During this process, the mucosa of the turbinates, which is responsible for the mucociliary clearance (MCC), is not damaged. Since the basic functions of the nasal cavity are dependent on the MCC, its preservation is important. Since the treatment of the tissues does not depend on the temperature of the working distal end, but solely on LFUS and its absorption by the vascular tissues, the resulting wave may be predictably limited in its reach and may be effectively extinct within a predetermined distance from the distal functional tip 118.

This cavitation-caused coagulation (as opposed to thermally-caused coagulation), phenomenon ensures a fined-grained control of the zone of action of the LFUS energy and, therefore, a highly predictable zone and volume of tissue coagulation. However, one problem encountered during operation of conventional low-frequency ultrasound devices is that of tissue adhering to the distal functional tip 118. If not accounted for and prevented, retraction of the distal functional tip 118 can cause damage, as adhered tissue may be pulled and torn from the nasal turbinates. This can result in heavy and sustained bleeding that can prove to be difficult to control. According to one embodiment, the shape of the handle 202 is intimately related to the safety and efficacy of the procedure to be carried out by the present ultrasound device. That is, by providing a handle 202 having a shape and features that assist the practitioner in avoiding tissue adhesions to the distal functional tip 118, the resulting outcomes are better than would be otherwise obtained. Indeed, the shape and features of the handle 202 enable the operator to move the distal functional tip 118 in a very specific sequence and/or pattern of movement. Indeed, to prevent such tissue adhesion effect and its related potential trauma to the interior structures of the turbinate, the shape and the ergonomics of the handle 202 are configured to enable the operator to cause the distal functional tip 118 to carry out a dual movement comprising a simultaneous reciprocating motion as suggested at 404 in FIG. 4 and circular or angular motion as suggested at 406 within the inferior turbinates 402, for example. This shape and form factor of the handle 202 according to one embodiment specifically enables the operator to perform turbinate reduction using a hand-held device in the safest and most efficacious manner—that is, while causing a simultaneous reciprocating and circular (or near circular or angular) motion at the distal functional tip 118 during at least part of the procedure.

Toward that end, one embodiment of the handle 202 and the ultrasound emitter 100 may be configured so as to cause the distal functional tip 118 to repeatedly trace a reciprocating path within the nasal turbinate and/or trace a circular or otherwise angular path therein. Such configuration frees the practitioner from having to manually move the distal function tip 118 back and forth with the turbinates and from having to move the distal functional tip 118 along a circular or angular path therein, thereby enabling a safe and efficacious performance of the tissue disintegration procedure. Building this functionality into the handle 202 and/or ultrasound emitter 100 simplifies the procedure and minimizes a possibility of a human error. Alternatively, according to one embodiment, the handle 202 and/or the ultrasound emitter may be configured so as to cause the distal functional tip 118 to repeatedly trace only the reciprocating path 404 within the nasal turbinate or only trace a circular or otherwise angular path 406 therein.

As also shown in FIG. 4, one embodiment comprises one or more depth of penetration markers 408 located on or near the distal functional tip 118. The depth of penetration markers 408 may be configured to enable the practitioner to distinguish one from other, with each depth penetration marker indicating a predetermined penetration depth. For example, the markers 408 may be colored, have a reflective surface or different surface texture, or may comprise one or more notches for ease of visualization. This is particularly useful when the procedure is carried out under the endoscopic guidance. Those of skill in this art may recognize that other depth of penetration modalities may be implemented. For example, other visual or audio indications may be provided, to assist the practitioner in gauging the depth of penetration of the distal functional tip 118 within the nasal cavity.

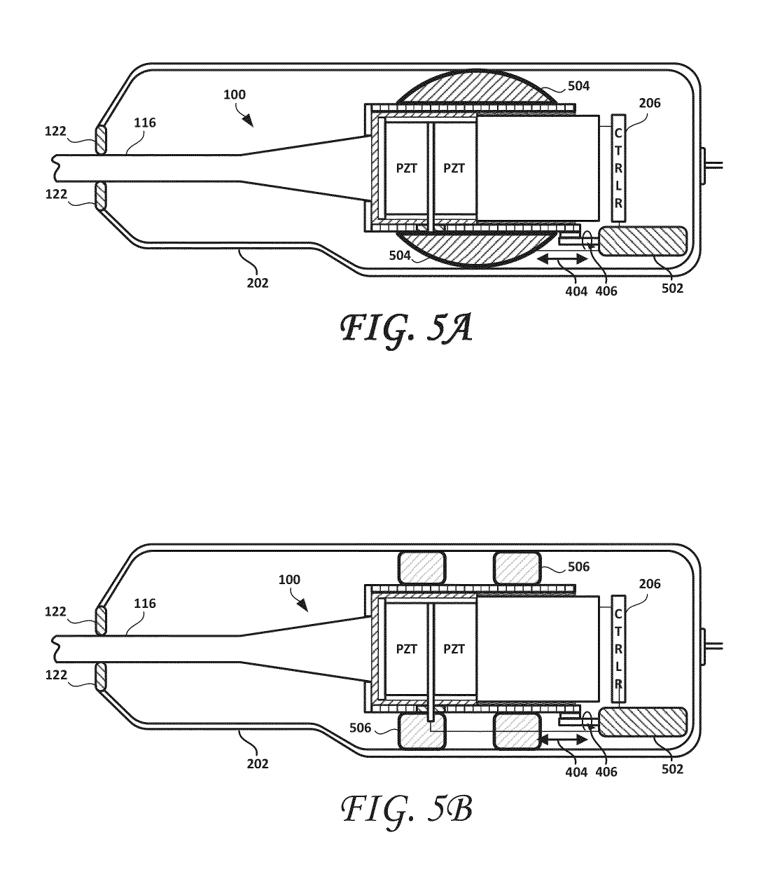

According to one embodiment, a movement generating mechanism such as, for example, a small eccentric motor and shaft assembly 502 may be coupled to the ultrasound emitter 100, as shown in FIG. 5A. This eccentric motor 502 may be configured to, in operation, impose a (e.g., planetary, reciprocating, angular motion or other vibrational) movement onto the distal functional tip 118. According to one embodiment, the ultrasound emitter 100 may be at least partially encased semi-rigidly within the handle 202. For example, to facilitate the reciprocal, circular, angular or vibrational motion imposed by the eccentric motor 502, a layer of rubber or other compliant and elastic medium may be disposed between the ultrasound emitter 100 and the handle 202. According to one embodiment, the ultrasound emitter 100 may be at least partially surrounded by a compressible material, such as ball or mass 504, within the handle 202. In this manner, if the shaft of the eccentric motor 502 is coupled to a rigid portion of the ultrasound emitter and the ultrasound emitter is coupled to the handle 202 via the mass 504, movement of the shaft of the eccentric motor 502 may cause the ultrasound emitter 100 to move slightly or vibrate within the handle 202, due to the compliance and elasticity of the rubber ball or layer 502. Such small movement or vibration may be along a single plane as suggested by arrows 404 or 406 or simultaneously along the two planes 404, 406. When the compressible material is configured as a ball, the ultrasound emitter 100 may be disposed within a through bore defined within the ball.

Alternatively, according to another embodiment, reference 504 in FIG. 5 may denote an inflatable sleeve. Such a sleeve may be at least partially filled with a saline solution of with silicone gel, for example. An inflation and deflation mechanism (not shown) may be provided to facilitate easy insertion and positioning of the LFUS emitter 100 in the handle 202. That is, the inflatable sleeve 504 may be deflated upon positioning the ultrasound emitter 100 within the handle 202 and thereafter inflated within the handle 202, to semi-rigidly affix the ultrasound emitter 100 within the handle 202 prior to use without, however, mechanically and acoustically coupling the ultrasound emitter 100 with a large mass. Advantageously, the rubber mass, inflatable sleeve or layer 504 does not mechanically or acoustically couple any significant mass to the ultrasound emitter 100 and does not significantly degrade the performance of the distal functional tip 118 through loss of acoustic energy or quenching of the resonance condition.

As shown in FIG. 5B, the ultrasound emitter 100 may be supported within the handle 202 by one or more compressibles, such as acoustically isolating annular supports 506. Such supports 506 may be surround the ultrasound emitter 100 without, however acting as a significant damper to the applied ultrasonic energy. For example, the acoustically isolating annular supports 506 may be a low mass, high density foam. Other structures and mechanism to support the ultrasound emitter 100 within the handle 202 may be devised by those of skill in this art.

According to one embodiment, the ultrasound emitter, the concentrator, the waveguide and the distal functional tip are integrated into a single, non-detachable assembly. This configuration eliminates the need to exchange functional waveguides intra-operatively, which reduces the procedural time and reduces the chance for human error. It also allows individual tuning with the unit's piezoelectric transducer's intrinsic frequency, avoids introducing any incompatibilities between the waveguide and the transducer, and eliminates a potentially troublesome joint. The handle 202 for the integrated unit may be single use and disposable. Alternatively, the handle 202 may be configured for multiple uses and may be sterilized in an autoclave, for example.

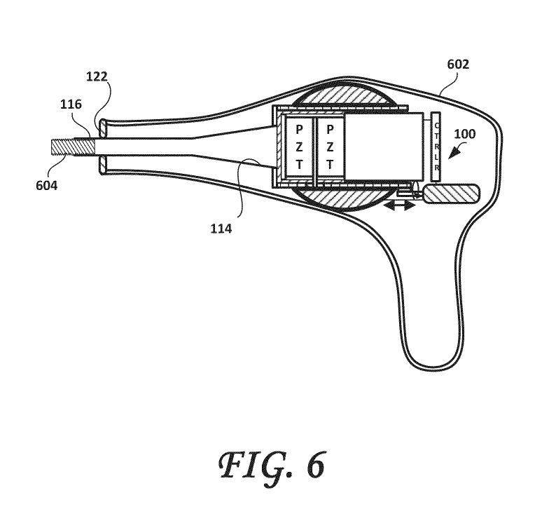

As shown in FIG. 6, the ultrasound emitter 100 may be disposed and encapsulated within an ergonomically-configured handle. One shape that lends itself well to the human hand is a so-called pistol-grip shape, as shown at 602. According to one embodiment, the rubber mass, layer or inflatable sleeve 504 and the hermetic collar 122 may be only points of contact between the ultrasound emitter 100 and the handle 602, so as to not mechanically and acoustically couple the ultrasound emitter therewith. FIG. 6 also shows aspects of another embodiment. Indeed, whereas FIGS. 1-5 show an integrated ultrasound emitter in which the handle, the ultrasound emitter, the concentrator, the waveguide and the distal functional tip are integrated into a single, non-detachable assembly, FIG. 6 shows an embodiment in which at least a portion of the cylindrical waveguide is detachable from the remaining assembly and exchangeable. To enable changing the distal portion of the cylindrical waveguide 116 and the distal functional tip 118, the cylindrical portion may include a threaded portion 604, to enable a mating waveguide and distal functional tip to be fitted thereto. This enables ready replacement of the distal functional tip 118 of the cylindrical waveguide 116 according to the procedure envisaged. A number of cylindrical waveguides with different distal functional tips may be provided, with each being field-replaceable within the office or the operating room setting. According to one embodiment, the conical concentrator 115 may also be swapped out for another concentrator having different acoustical characteristics.

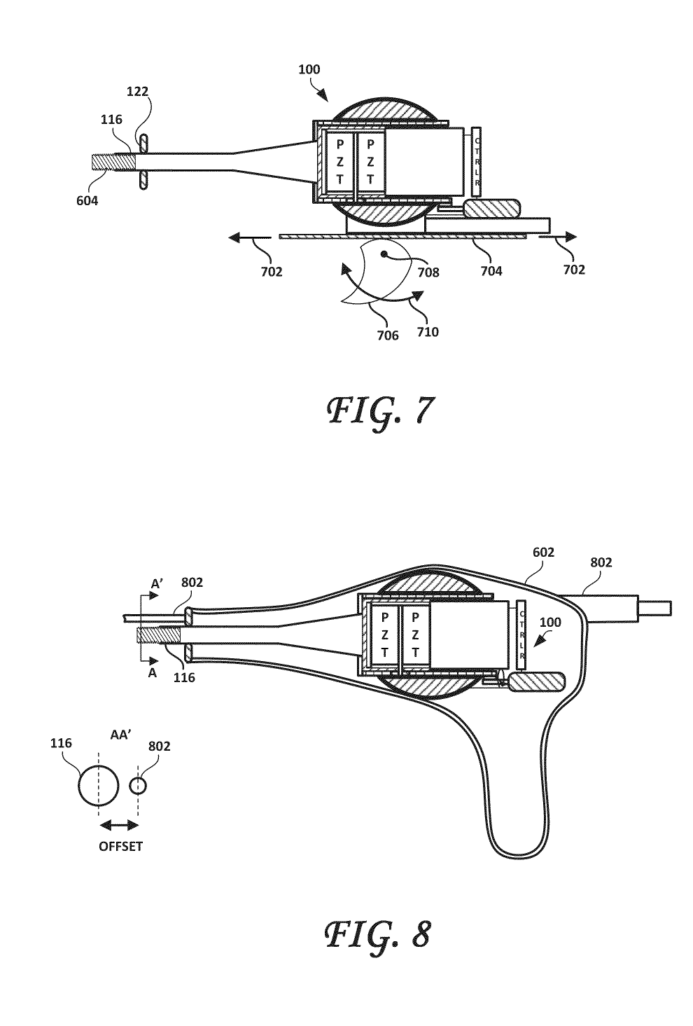

FIG. 7 is a diagram of an ultrasonic emitter 100 comprising a reciprocating mechanism, according to one embodiment. To provide a more pronounced longitudinal movement (i.e., in a direction 702 parallel to the longitudinal axis of the cylindrical waveguide 116, for example), the ultrasound emitter 100 may be mounted on or otherwise coupled to one or more rails 704. A trigger 706 may be provided, as shown in FIG. 7. According to one embodiment, the trigger 706 may be coupled to a motor configured to drive the ultrasound emitter 100 along the rail 704. Alternatively, according to one embodiment, the trigger 706 may be coupled to the rail 704 so as to form a rack and pinion assembly. That is, when the user depresses the trigger 706, the trigger 706 may pivot about its pivot point 708 as suggested at 710, and the teeth defined on the superior surface thereof in contact with the rail 704 may engage corresponding structures on the rail 704 to drive the assembly forward. The trigger 706 may be spring loaded, to bias the assembly back to its initial position. It is understood that those of skill in this art may devise other mechanisms and methods for driving the ultrasound emitter 100 in a reciprocating motion, and all such mechanisms and methods are deemed to be included herein. Including such reciprocating motion functionality ensures that the operator's hands may remain immobile, facilitates smooth motion and helps the practitioner to precisely control the depth of penetration of the distal functional tip 118 into the nasal cavity. The embodiment of FIG. 7 is also well suited to the pistol-grip type of handle shown in FIG. 6. According to one embodiment, a trigger locking mechanism may be provided to lock the trigger 706 in position, which may also limit the number of times the distal functional tip 118 is inserted in the turbinate.

FIG. 8 is a diagram of an ultrasonic emitter comprising offset visualization optics, according to one embodiment. For better visualization of the target zone, one embodiment comprises endoscopic optics 802 coupled to or incorporated within the handle 602. As shown in FIG. 8, the optics 802 advantageously may be medially offset relative to the longitudinal axis of the ultrasound waveguide 116 and of the distal functional tip 118, as shown by the cross-sectional view thereof along AA′. This configuration allows for the visualization to be unencumbered by ultrasound waveguide 116 and the distal functional tip 118. The optics 802 may be detachably coupled to the handle 602 or may be incorporated therein.

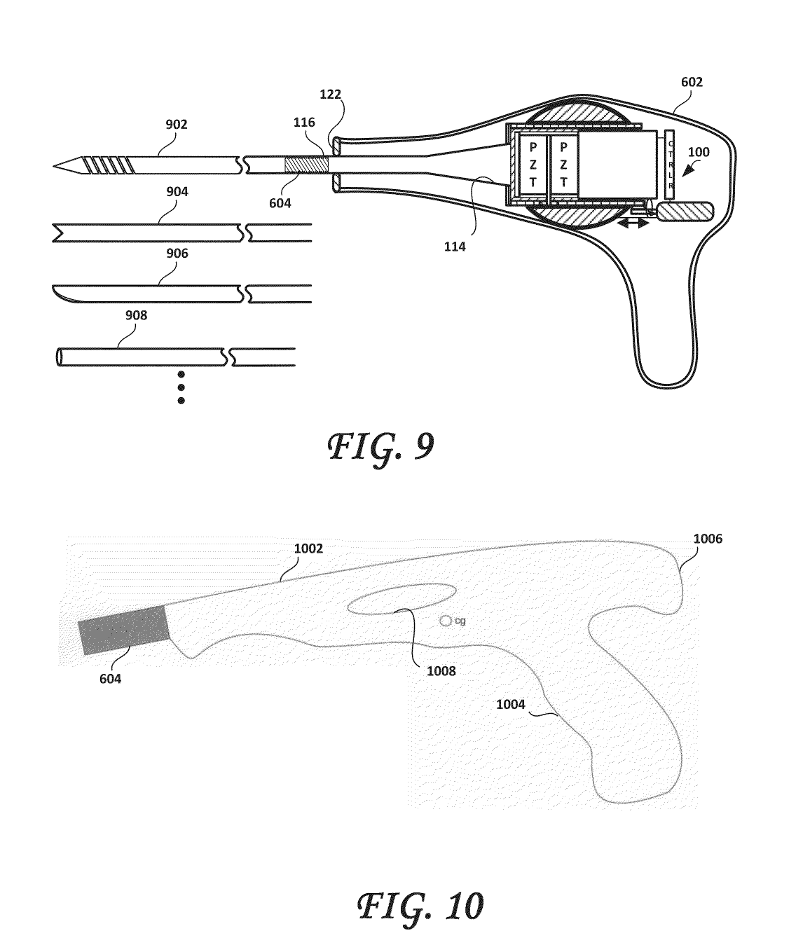

FIG. 9 is a diagram of an ultrasonic emitter configured for selective attachment of different cylindrical waveguides with distal functional tips, according to one embodiment. As shown, a variety of cylindrical waveguides 902, 904, 906 may be removably fitted to the ultrasound emitter 100, through engagement with the threaded portion 604. Different distal functional tips, coupled with the application of different ultrasonic energies, may be suited for performing different procedures. For example, the distal functional tip of cylindrical waveguide 902 is configured for turbinate tissue disintegration. The distal functional tip of cylindrical waveguide 904 is configured for bone spur removal. The distal functional tip of cylindrical waveguides 906 and 908 may be configured for epitasis. Other cylindrical waveguides comprising different distal functional tips may also be provided, as those of skill in this art may recognize.

FIG. 10 is a diagram of a handle of an ultrasonic emitter, according to one embodiment. As shown, the handle 1002 may have a generally pistol-grip shape, with a substantially central center of gravity (cg). In this embodiment, the pistol grip handle 1004 and the proximal projection 1006 provide a great deal of space for control circuitry, motors and others of the constituent components of the present low frequency ultrasound device. A switch 1008 may be provided in an ergonomically suitable position, to enable the operator to selectively turn the present low frequency ultrasound device on and off

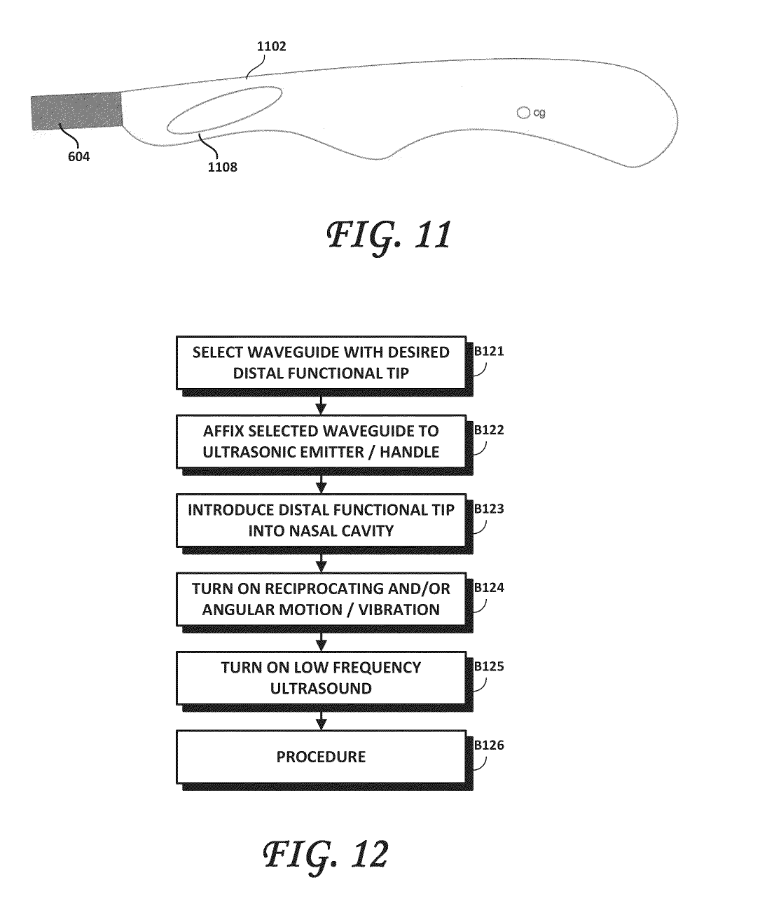

FIG. 11 is another diagram of a handle of an ultrasonic emitter, according to one embodiment. As shown therein, the handle 1102 may have a more streamlined aspect, with the center of gravity (cg) moved proximally, as compared to the embodiment depicted in FIG. 10, to balance out this distally-heavy configuration. A switch 1108 may be provided in an ergonomically suitable position, to enable the operator to selectively turn the present low frequency ultrasound device on and off

FIG. 12 is a flowchart of a method of carrying out a procedure within a nasal cavity, according to one embodiment. As shown at block B121, the practitioner may select a waveguide with a desired distal functional tip, as shown at 902, 904 and 906 in FIG. 9. The waveguide with the selected distal functional tip may then be affixed to the ultrasonic emitter 100 and/or the handle, as called for, at block B122. Block B123 calls for the practitioner to introduce the distal functional tip of the selected and affixed waveguide into the patient's nasal cavity. To prevent tissue adhesion, the practitioner may then turn on and/or otherwise activate the mechanism or assembly that enables the distal functional tip to undergo a reciprocating, angular or vibrational movement, as shown at B124. At block B125, the low frequency generator may be activated, to cause the ultrasound emitter 100 to generate low frequency ultrasound energy at the distal functional tip, while the latter is moving in a reciprocating, angular or vibrational movement pattern. Lastly, the practitioner may then carry out the intended procedure as shown at B126, such as microdebridement, epitasis, turbinate reduction or polyp removal, to name but a few possible procedures.

Surgery of the head and neck requires meticulous techniques and instrumentation that enables precise tissue handling. The nose, sinuses, pharynx, larynx and ear all lie in highly vascular regions, adjacent to functional musculature and cranial nerves. Removal of disease should be carried out with strict control of bleeding, and without destroying neighboring structures or causing disfigurement. Precision on a layer by layer basis is the bedrock of efficient and non-crippling surgery in this region.

Low frequency ultrasound, as generated by emitter 100, enables fine cutting, coagulation and controlled tissue ablation. The embodiments of ultrasound devices described and shown herein harness the power of surgical ultrasound for otolaryngology, as detailed hereunder. It is to be noted that the below-detailed procedures constitute but a fraction of the procedures to which the present embodiments may be applied. Moreover, it should be recognized that suction may be applied to the operative site, contemporaneously with the application of ultrasound energy or thereafter, to evacuate the site and keep it clear of debris, to aid the surgeon's visualization. According to embodiments, such suction may be integrated with the ultrasound emitter or may be separate therefrom.

Nose

The nasal cavity, the paranasal sinuses, the nasal septum and the external nose are rich and anastomotic and have the potential to bleed heavily. The eyes and brain lie immediately adjacent to the nasal anatomy and are subject to potential catastrophic injury if the boundaries of the nasal and sinus anatomy are transgressed. Minimally invasive approaches are now the standard, and surgical access through the nostril requires delicate and precise instrumentation.

Examples of intranasal surgical procedures (shown at Block B126 in FIG. 12) that may advantageously be carried out with an ultrasound emitter according to the present embodiments include turbinate reduction and turbinate removal, septoplasty, control of epistaxis, polypectomy, synechia removal, sinusotomy and sinusectomy, sphenopalatine artery ligation, endoscopic transnasal approach to the skull base, closure of cerebrospinal fluid leak, orbital decompression and hypophysectomy. Examples of extranasal procedures include external approach to ethmoid/maxillary (Caldwell Luc) and frontal sinuses, anterior and posterior ethmoid artery ligation and open rhinoplasty.

Indeed, the following examples illustrate the effects of deploying embodiments of the present ultrasound devices for nasal procedures and their advantages.

Turbinate reduction for nasal obstruction: In an office setting under topical local anesthesia with the patient seated in a chair, Block B126 of FIG. 12 may be carried out by inserting a selected distal functional tip 118 into the fleshy anterior end of the inferior turbinate, to depth of about 12 mm (for example), the distal functional tip energized and thereafter removed after about 10 seconds, for example. Bleeding and pain may be minimized and the patient may be discharged within minutes. Within a day, the patient may experience slight swelling and obstruction, which may be treated with a small dose of acetaminophen. Within two weeks, there is substantial beneficial reduction of the turbinate with a widened nasal passage. Advantageously, the procedure may be carried out in an office setting, with minimal anesthesia, rapidly and a non-intrusive post-operative course. In contrast, other treatment modalities (radio frequency, for example) cause scarring of the nasal mucosa leading to a dry, crusting nasal cavity.

Septoplasty and septal spur removal: In an office setting under local anesthesia and seated in an operating chair and using a nasal endoscope for illumination and visualization in this narrow space, Block B126 of FIG. 12 may be carried out by using a selected distal functional tip 118 to incise and elevate the nasal mucosa in a bloodless fashion. The selected distal functional tip 118 may then be caused to disintegrate the cartilage and/or bone without opening the mucosa on the opposite side of the cartilage or bone. A small tunnel created may be collapsed and may thereafter be held in place by nasal staples, which may be selected to dissolve in a matter of days. No packing need be used. Advantageously, the procedure may be rapidly carried out in an office setting, with local anesthesia, with minimal bleeding. Significantly, use of the present emitter 100 in septoplasty procedures does not endanger the mucosa on the far side of the cartilage or bone resection. Bleeding, permanent through and through perforation and the need for nasal packing minimized or obviated.

Caldwell Luc canine fossa approach to the maxillary sinus: In an office setting, seated in an operating chair and under local anesthesia, Block B126 of FIG. 12 may be carried out by using a selected distal functional tip 118, such as an ultrasonically activated scalpel, may be used to bloodlessly incise the mucosa down to the bony anterior wall of the sinus. Thereafter, another selected distal functional tip may be attached and the bone may be painlessly incised in a controlled manner with full visualization of the infraorbital nerve, thus preserving this structure. The bone flap may be elevated, still attached to the overlying mucosa. After endoscopically guided removal of the maxillary sinus lesion, the mucosa/bone flap may be replaced and sutured in or plated place. The bone flap heals in place, as it is fully vascularized. Advantageously, this quick procedure B126 may be carried out in an office setting, under local anesthesia, with minimal bleeding. In particular this procedure B126 may be carried out without substantial bleeding, which allows for full visualization of the infraorbital nerve and thus helps ensure its preservation. In addition, unlike the conventional usual Caldwell Luc procedure, the bone flap is not destroyed and full integration of the bone back into the maxilla is possible. An advantage here is that cutting through the bone can be done so precisely the underlying mucosa can be spared.

External rhinoplasty: In surgical center setting, under local plus sedation anesthesia or general anesthesia, Block B126 may be carried out by bloodlessly elevating the skin of the nose from the underlying bony and cartilaginous skeleton, using a selected distal functional tip 118. In full view, the distortions of this skeletal structure may be modified with selected distal functional tips 118 to finely and bloodlessly transect or resect for functional and cosmetic purposes. The skin may then be returned and carefully sutured. Advantageously, the minimal or lack of bleeding and the fine cutting with little collateral damage to both soft and hard tissues leads to a more precise rearrangement of the skeletal parts, ease of closure and less swelling afterward.

Control of epistaxis: In an office chair, under local anesthesia, Block B126 of FIG. 12 may be carried out by undermining the site of the bleeding in the nasal cavity with the ultrasound emitter 100 configured with a selected a distal functional tip 118. The ultrasound causes coagulation of the open end of the vessel and intravascular coagulation without leaving an open ulcer, as would otherwise be seen from RF electrocoagulation or chemical cautery with silver nitrate. Advantageously, such a procedure offers rapid healing, less chance for re-bleed and reduction in the chance of aggravating septal perforation from this benign problem.

Transnasal endoscopic ethmoidectomy and polyp removal: In a surgery center, operating room or [clinic (or office)], using local anesthesia and an endoscope, Block B126 of FIG. 12 may be carried out using an embodiment of the ultrasound emitter 100 equipped with a selected distal functional tip 118 to reduce or resect polyps bloodlessly to open the nasal passage. The lateral wall of the nasal passage may be incised through the mucosa and bone to expose the lining of the ethmoid, maxillary or sphenoid sinus lining. This lining may then be selectively treated to reduce polypoid changes and to sterilize the surface, leaving access to the nasal passage. The bloodless entry allows specific dissection to targeted sinus cavities. When Block B126 is carried out under stereotaxic guidance, clear bloodless visualization of the medial orbital wall and the skull base may be accomplished with accuracy and safety. Advantageously, precision surgery to these tight areas that are bounded by the eye, optic nerve and brain and its dural coverings makes surgery to open all of the tiny sinus cavities much less dangerous and much more thorough. The postoperative bleeding may be greatly decreased and may not require hemostatic foreign materials as well as obstructive nasal packing, all of which must eventually be removed. An advantage of ultrasound as it cuts bone is the preservation of soft tissue on the other side of this bone. In the case of the skull base, the bone can be cut without injury to the dura.

The combination of precise cutting, tissue disintegration and narrow field coagulation expands LFUS to other areas of the head and neck.

Nasopharynx:

The nasopharyngeal walls consist of the mucosal/muscular posterior and lateral walls, the choanae that are the entrances to the nasopharynx from the nose, and the soft palate. Deep to the posterior wall is the clivus and the first two vertebrae. In the lateral wall is the Eustachian tube cartilage and orifice. The adenoid tissue protrudes from the mucosa. Each of these structures is a target for various procedures.

Procedures of and through these walls of the nasopharynx are numerous, ranging from adenoidectomy, to opening of choanal atresia, to approaches to the clivus (for tumor) and first three cervical vertebrae (for degenerative disease and trauma), to soft tissue pedicle flaps (for reconstitution of nasopharyngeal function) and to soft palate procedures (for the treat of obstructive sleep apnea and snoring). LFUS devices according to embodiments allow each of these to be carried out in the operating room with more precision and with markedly less bleeding.

Of special interest is that with topical and well placed conduction anesthesia, (injection of local anesthetics) some of these procedures may be accomplished in an office setting. Two examples of such procedures that are well adapted to being carried out in the office include reduction of adenoid tissue affecting Eustachian tube function (leading to otitis media) and the treatment of obstructive sleep apnea and snoring.

Removal of adenoid tissue is a matter of tissue disintegration. As the LFUS wave guide according to embodiments is thin as seen in the nasal procedures, these may be endoscopically guided through the nose and posterior choanae to remove adenoid tissue in a very precise manner, as an office procedure. This friable tissue may be disintegrated with excellent hemostatic control without injuring the Eustachian tube structures. The process may be carried out in a quick and painless manner.

Soft palate reduction to control obstructive sleep apnea and snoring also is amenable to treatment using the present embodiments. Such procedures may be carried out transorally. With an endoscope to achieve precise placement of the present waveguide, resection may be carried out on an alert, awake patient. With the patient able to cooperate, it is possible to remove the posterior rim of the soft palate and the uvula in a fashion that will markedly reduce if not eliminate the conventionally-encountered problems of nasopharyngeal escape. Again, topical and conduction anesthesia may be used very effectively, making such procedures amenable to office implementation utilizing the present embodiments.

Throat

Areas of obvious interest in the throat include the tonsils and adenoids, the base of tongue, pharyngeal walls and larynx. These areas are exposed with endoscopes and microscopes to bring illumination and magnification. The latter are advantageous as precision dissection can reveal significant vasculature that can be dealt with before cutting that fills the small field with blood. In some situations, the small collateral damage using an ultrasound emitter 100 and selected distal functional tips 118 according to embodiments allows a very much more precise resection than possible even with a narrow beam (0.3 mm) CO2 laser.

Tonsillectomy: Block B126 in FIG. 12 may comprise a tonsillectomy procedure that may be carried out in a surgery center or in a hospital operating room venue. The procedure may be carried out with general or under local anesthesia. A selected distal functional tip 118 may be deployed to disintegrate the tonsil by direct application. Interestingly, the ultrasonic disintegration action differentiates between the lymphoid tissue of the tonsil and the blood vessels so that individual blood vessels can be coagulated, with minimal blood loss. Advantageously, intracapsular tonsillectomy has been shown to be effective in removing the lymphoid tissue of a tonsil affected by chronic infection (thought to be associated with and due to biofilm growth in the depths of the tonsil crypts). The lack of dissection outside of the tonsil capsule to which the pharyngeal constrictor muscles attach using an embodiment means more rapid recovery of swallowing and overall healing. As the blood vessels can be individually coagulated, surgical blood loss is held to a minimum. The resection can be done in a manner that does not breech the tonsillar capsule

Tongue base, laryngeal and pharyngeal wall cancer resection: Embodiments of the present ultrasound emitter 100 and distal functional tips 118 enable effective microscopic transoral robotic removal of tongue base, laryngeal and pharyngeal wall cancers, which are increasingly recognized today as an effective surgical approach to cancers in these areas. Embodiments enable the post-operative course to be much easier for the patient than is the case with conventional open procedures, and allow rapid discharge from the hospital, early alimentation and avoidance of tracheostomy all with cure rates comparable or better than external approaches. In contrast to conventional treatment modalities using, for example, CO₂ lasers, the use of ultrasound emitter 100 according to embodiments leads to comparative reduction of the collateral damage conventionally associated with such procedures and a more bloodless dissection. Moreover, the use of the present ultrasound emitter 100 can significantly reduce negative postoperative effects, with improved visualization equaling or bettering the dissection results of conventional techniques. In addition, use of the present embodiments provides a direct tactile feedback of the affected tissue to the practitioner that cannot be achieved with touchless lasers. This does not mean, however, that ultrasonic emitters according to embodiments cannot be used for external procedures, where dissection can be more precise than possible with cold knife or RF electrocautery as used often today. Advantageously, use of the present embodiments enables a more precise, less bloody surgery allowing exacting dissection, with good therapeutic results and faster time to discharge. There is less need for tracheostomy as swelling is decreased. Pain is less and thus requiring less medication. As hospital inpatient time is decreased, so too is the risk of contracting hospital-borne infections. Such results may be achieved due to reduced bleeding, better visualization and reduced collateral damage occasioned through the use of embodiments.

Laryngeal/vocal cord benign lesion: In either an office, surgery center or hospital setting under local or general anesthesia, Block B126 of FIG. 12 may comprise transoral removal of intralaryngeal lesions. The precision and lack of heating resulting from use of embodiments of the ultrasound emitter 100 and selected distal functional tips 118 enables the removal of lesions, without damage, of the delicate underlying tissues such as the lamina propria of the vocal cord that is needed for slippage of the vocal cord mucosa over the underlying vocal ligament and thyroarytenoid muscle. Conventionally, such procedures are carried out with a cold knife or a laser. The bleeding occasioned by the use of such conventional tools must be controlled with intra mucosal injection of epinephrine containing solutions to cause vasoconstriction. This process distorts the lamina propria. The actual dissection must stay out of the laminal propria for the best vocal results. Use of an embodiment can allow this to happen as injection is not needed to control bleeding. As there is no heat, as is the case with CO2 or holmium lasers, the all-important lamina propria may be preserved and with it the clarity of the sound produced by the vocal cords.

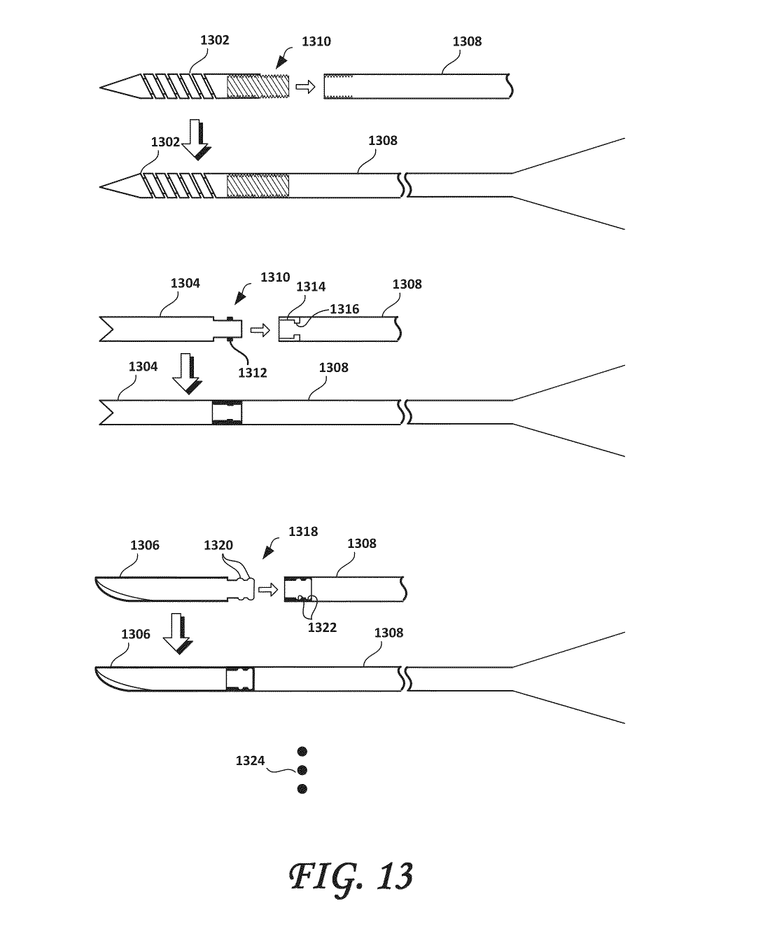

In contrast to FIGS. 1-5, which show embodiments of an integrated ultrasound emitter in which the handle, the ultrasound emitter, the concentrator, the waveguide and the distal functional tip are integrated into a single, non-detachable assembly, and in contrast to FIGS. 6-11, which show embodiments in which at least a portion of the cylindrical waveguide is detachable from the remaining assembly and exchangeable, FIG. 13 shows a further embodiment that comprise detachable distal functional tips. FIG. 13 shows a variety of detachable distal functional tips configured for use with the present ultrasound emitter, according to embodiments. According to one embodiment, the distal functional tip may be detachable from the cylindrical waveguide. FIG. 13 shows a number of distal functional tips 1302, 1304 and 1306, each of which may be selectively attached and detached from the cylindrical waveguide 1308 of the ultrasound emitter—between procedures or during the course of a single procedure. Even when constructed with high quality titanium and/or alloys thereof, the distal functional tips may degrade over time. Therefore, it is prudent to replace the distal functional tips 1302, 1304, 1306 after a predetermined number of uses. For safety and efficacy purposes, therefore, the distal functional tips may be configured for a limited number of uses, after which they may be replaced. For example, the distal functional tips 1302, 1304 and 1306 may be configured to be single use and disposable. Alternatively, such distal functional tips may be configured for a greater, but limited number of discrete uses. Replacing the distal functional tips after a predetermined number of uses prevents or substantially reduces the chances that particles (of Ti, for example) may become dislodged from the distal functional tip and become accelerated within the nasal cavity by the ultrasound-induced cavitation. Also, degradation of the distal functional tips undesirably changes the ultrasound resonance characteristics thereof.

Notwithstanding, the distal functional tips 1302, 1304, and 1306 may be configured with a variety of attachment mechanisms and structures to enable them to couple to the waveguide 1308 in a manner that is both mechanically secure and acoustically transmissive, so that the applied ultrasound energy is not substantially dampened at the interface between the cylindrical waveguide 1308 and the detachable distal functional tip 1302, 1304, 1306. Such coupling requires a tight mechanical coupling and high metal-to-metal surface contact between the coupled elements. For example, the distal functional tip 1302 may couple to the cylindrical waveguide by a male structure and a mating female structure. For example, the male and female structures may be a bolt and threaded socket assembly, as shown at 1310 in FIG. 13. The distal functional tip 1302, therefore, may be screwed onto the cylindrical waveguide 1308. Either the cylindrical waveguide 1308 or the distal functional tip 1302 may be configured with either the male or the mating female structure. Alternatively, as shown at 1310, the distal functional tip 1304 may be configured with bayonet-like projections 1312 and the cylindrical waveguide may be fitted with corresponding channels 1314, thereby enabling the distal functional tip 1304 to be inserted within the cylindrical waveguide 1308 by guiding the bayonet-like projections 1312 into the corresponding channels 1314 and rotating (e.g., an eighth of a turn, for example) the distal functional tip 1304 with respect to the cylindrical waveguide 1308, to lock the bayonet-like structures within the right-angled blind channel 1306 within the cylindrical waveguide 1308. Other attachment mechanisms are possible. For example, a snap fit connection may also be envisaged, as shown at 1318. The distal functional tip 1306 may define one or more convex surfaces 1320 and the cylindrical waveguide 1308 may define one or more corresponding mating concave surfaces 1322. When the distal functional tip 1306 is fitted to the cylindrical waveguide 1308, the respective convex and concave surfaces 1320, 1322 may be pushed into intimate contact with one another, thereby securing the distal functional tip 1306 to the cylindrical waveguide 1308. Alternatively and as shown in FIG. 13, the distal functional tip 1306 may define the concave surfaces and the cylindrical waveguide 1308 may define the convex surfaces. Other snap-fit mechanisms may be readily implemented, as those of skill in this art may recognize, as suggested at 1324. Indeed, other attachment structures may be devised within the context of the present embodiments.

The detachable distal functional tips shown in FIG. 13 are exemplary and represent but a fraction of the universe of possible distal functional tip configurations. Indeed, variables such as the nature of the procedure envisaged, the applied ultrasound frequencies and amplitudes, the fibrous or vascular nature of the soft tissues to be treated, among others, may affect the geometry of the distal functional tip. For example, the shape, size, geometry, bending angle, length, thickness, materials, presence or absence of edges and/or points, the surface roughness and the like are all variables that may be varied to achieve the desired results.

According to one embodiment, the coefficients of expansions of the material(s) used for the cylindrical waveguide 1308 and for the distal functional tip may be used to good advantage. For example, the cylindrical waveguide 1308 may be heated before a selected distal functional tip is coupled thereto. Such heating may cause at least the distal portion of the cylindrical waveguide 1308 to expand, thereby creating increased clearance and easing the insertion of the selected distal functional tip therein. As the cylindrical waveguide 1308 cools, it may contract, thereby tightly coupling with the inserted distal functional tip with greater force than would be otherwise the case in the absence of such heating. Alternatively or in combination, the distal functional tip may be cooled before insertion/affixation to the cylindrical waveguide 1308. Such cooling may cause the distal functional tip to contract, thereby again easing its insertion or coupling to the cylindrical waveguide 1308 and ensuring a good acoustical transmission path for the applied ultrasound energy.

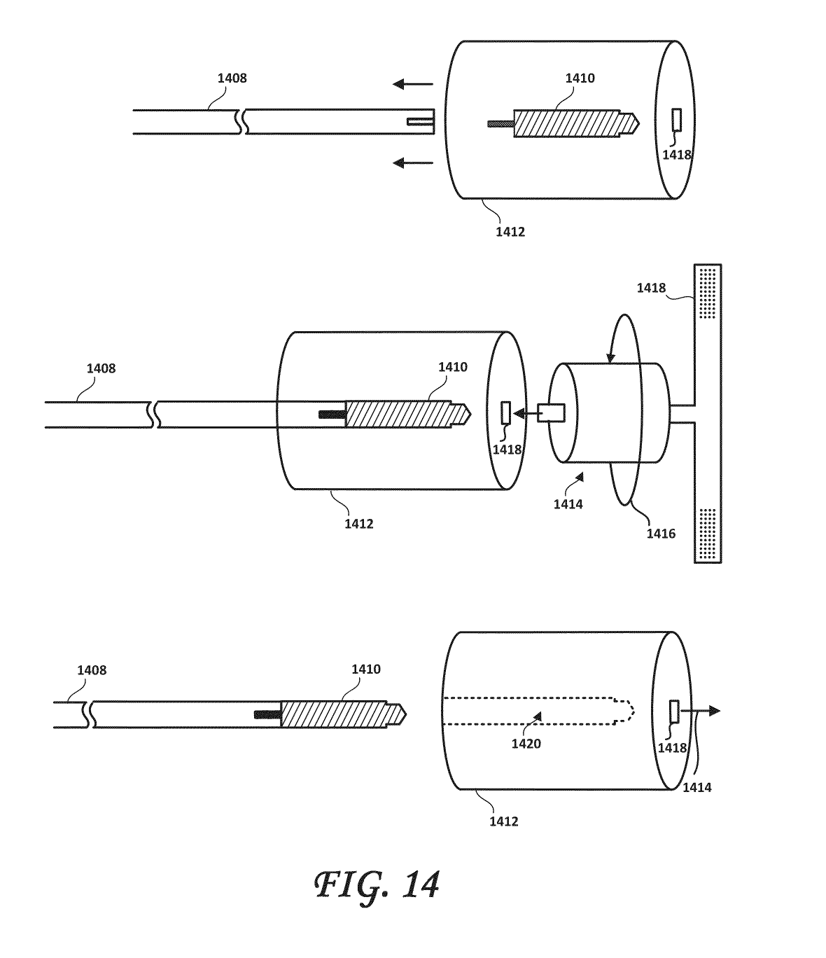

FIG. 14 shows methods and structures for attaching a detachable distal functional tip to a cylindrical waveguide of an ultrasonic emitter, according to embodiments. Reference 1408 denotes the distal end of the cylindrical waveguide of the ultrasound emitter 100. Reference 1412 denotes a connecting tool, according to one embodiment. The connecting tool 1412 may be configured to enable the secure and safe affixation of a selected distal functional tip 1410 to the cylindrical waveguide 1408. The connection tool 1412 may be configured, for example, as a cylinder or other shape comprising an internal surface defining a distal functional tip-shaped void 1420 therein. A distal functional tip 1410 may be disposed within the void 1420 in such a manner as to maintain the distal functional tip 1410 secure therein and to enable the distal functional tip 1410 to be screwed on or otherwise securely coupled to the cylindrical waveguide 1408. As shown at 1414, to couple the distal functional tip 1410 to the cylindrical waveguide 1408 with the proper amount of torque, a limited slip torque wrench 1414 may be used. The practitioner may apply the connection tool 1412 with enclosed distal functional tip 1410 to the distal end of the cylindrical waveguide 1408 such that the coupling features of each are aligned. The limited slip torque wrench 1414 may then be applied to a fitting 1418 within the connection tool 1412 and the T-handle 1418 may then be rotated to apply torque to the connection tool 1412. When a pre-set torque value is reached, the limited slip torque wrench 1414 may slip (with an audible indication, for example) and may automatically reset itself, thereby limiting the amount of torque applied by the connection tool 1412 to the cylindrical waveguide-distal functional tip assembly. As shown in FIG. 14, the limited slip torque wrench 1414 may then be removed from the fitting 1418 and the connection tool 1412 may be removed, leaving the cylindrical waveguide-distal functional tip assembly ready for the intended procedure. It is to be noted that the connection tool 1412 with the encapsulated or captured distal functional tip 1410 may be cooled prior to insertion on the cylindrical waveguide 1408. Thereafter, as the now-coupled distal functional tip 1410 warms to ambient temperature, the material thereof may expand, further ensuring a secure connection between the distal functional tip 1410 and the cylindrical waveguide 1408. In addition to providing a secure and consistent coupling, the connection tool 1412 ensures the practitioner's safety while handling potentially sharp distal functional tips, thereby reducing the chance of injury. According to one embodiment, the connection tool 1412 and captured distal functional tip 1410 may be sterilizable.

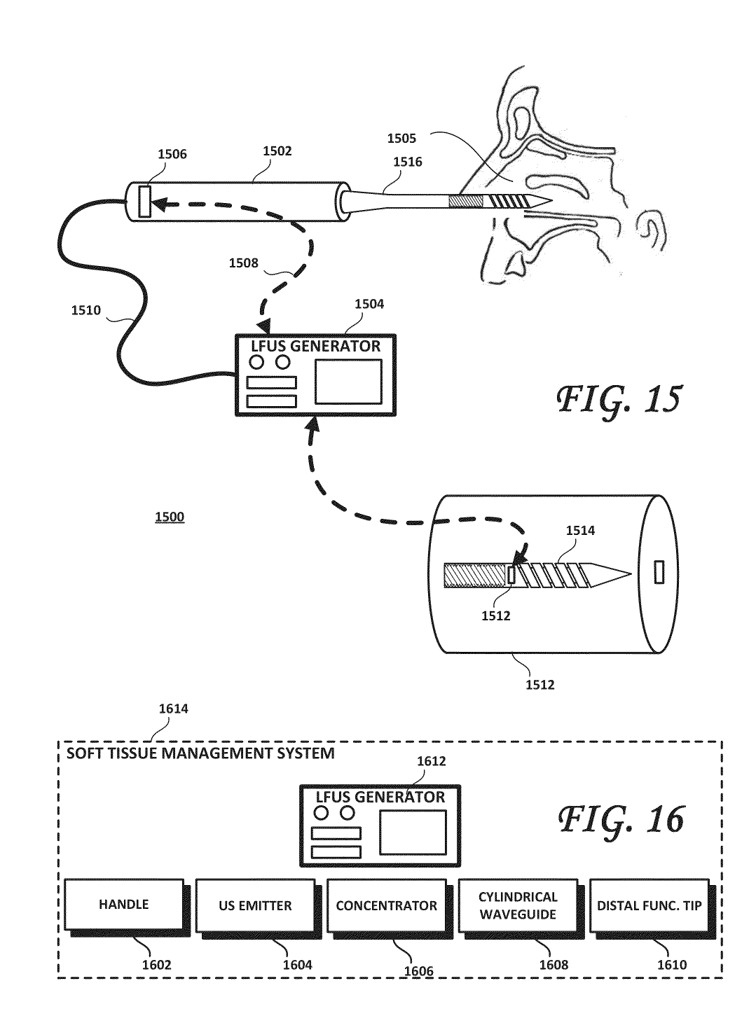

FIG. 15 shows a soft tissue management system 1500 according to one embodiment. As shown, an ultrasound device 1502 according to one embodiment is shown in use, with its distal functional tip inserted within the nasal turbinates 1505 of a patient. The ultrasound device 1502 (selectively configured according to FIGS. 1-14) may be coupled to a low frequency ultrasound generator 1504, configured according to, for example, reference 204 in FIG. 2. The coupling, shown at 1510, may be effective to transmit the electrical signals, generated by the low frequency ultrasound generator 1504, to be applied to the piezoelectric transducers within the ultrasound device 1502 for the generation of low frequency ultrasound energy at the distal functional tip thereof. According to one embodiment, the ultrasound device 1502 may comprise a communication unit 1506 that may be configured to communicate with the generator 1504, as suggested by reference 1508. Moreover, a communication device 1512 may be embedded or otherwise contained or encapsulated within the distal functional tip 1514. The communication unit 1506 and the communication device 1512 may communicate with one another or with the low frequency ultrasound generator 1504. For example, the generator 1504 may communicate with the communication unit 1506 to determine the number of times the distal functional tip has been used in a procedure. The generator 1504 may then, responsive to a signal from the communication device 1512, disallow further uses of the distal functional tip if the later has already been used a predetermined number and should be replaced. Toward that end, each distal functional tip 1514 may store or be associated with a unique identifier. The communication device 1512 may, for example, comprise a passive (wired or wireless) device that may polled or interrogated by the generator 1504, which may keep a running count of uses of the distal functional tip in a non-volatile memory. For example, the communication device 1512 may comprise a radio frequency identification device (RFID), a near-field communication (NFC) device or some functionally similar technology. Similarly, the generator 1504 may be configured to communicate with the ultrasound emitter 1502 and/or the distal functional tip 1514 and exchange information therewith. For example, the communication unit 1506 may communicate with the communication device 1512 in the distal functional tip 1514 and report to the generator 1504 which kind of distal functional tip is currently coupled to the cylindrical waveguide 1516. Based on this information, the generator 1504 may reconfigure the electrical signals to be applied to the piezoelectric transducers within the ultrasound device 1502, depending upon which distal functional tip 1514 is currently coupled to the cylindrical waveguide 1516. Such communication functionalities between the generator 1504, the ultrasound device 1502 and the detachable distal functional tip 1514 ensures a consistent and safe application of ultrasound energy to the distal functional tip 1514, dependent upon the type of procedure envisaged by the physician. For example, the generator 1504 may adjust the resonant frequency, amplitude and/or on/off duty cycle (for example) of the applied ultrasound energy dependent upon the procedure being carried out and depending upon the type, shape and structure of the distal functional tip 1514 currently coupled to the cylindrical waveguide 1516 and/or based upon the physiology of the tissue being acted upon.

FIG. 16 is a block diagram of a soft tissue management system, illustrating the selective modularity of the constituent components thereof, according to embodiments. As described herein above, the ultrasound device of the present soft tissue management system may be fully integrated, in that the handle, ultrasound emitter, concentrator, cylindrical waveguide and functional distal tip may be configured as a single integrated assembly, with no or few detachable parts. Alternatively, one or more of these constituent components may be configured to be detachable from the remaining components. For example, the distal functional tip 1610 may be detachable (and thus replaceable) from the remaining, integrated, assembly. Likewise, the cylindrical waveguide 1608 (with an integrated distal functional tip 1610 or configured to couple to detachable distal functional tips 1610) may be detachable from the concentrator 1606. Likewise still, the concentrator 1606 may be configured to be detachable from the ultrasound emitter 1604 (an may be configured with an integrated cylindrical waveguide 1608 or with a detachable cylindrical waveguide 1608 which, in turn, may comprise an integrated distal functional tip 1610 or may be configured to couple to detachable distal functional tips 1610). Similarly, the handle 1602 may be integrated with the ultrasound emitter 1604 or may be detachable therefrom. Therefore, it should be noted that each of the constituent components 1602, 1604, 1606, 1608 and 1610 of the present soft tissue management system may be detachable from the other components thereof, and thus replaceable and/or individually sterilizable. Alternatively, one or more of the components 1602, 1604, 1606, 1608 and 1610 may form an integrated assembly (e.g., an assembly that is not designed to be detachable into sub-components) with others of the components 1602, 1604, 1606, 1608 and 1610 and the low frequency ultrasound generator 1612 (and optionally the connection tool 1512 of FIG. 15) to form the present soft tissue management system 1614.

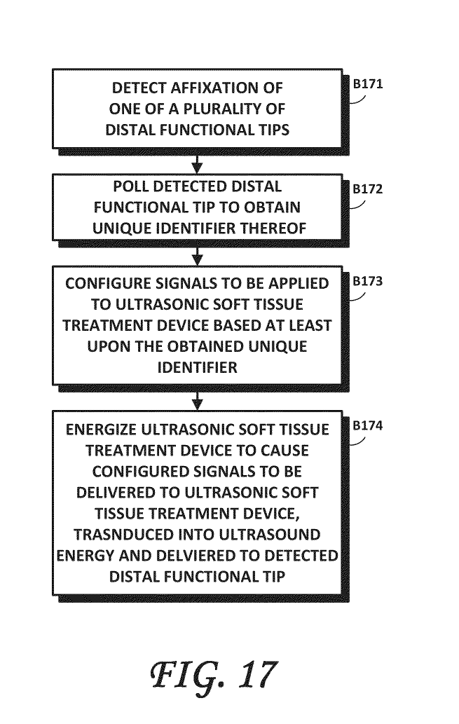

FIG. 17 is a method of operating an ultrasonic soft tissue treatment device, according to one embodiment. As shown, Block B171 calls for detecting an affixation, to the ultrasonic soft tissue treatment device, of one of a plurality of distal functional tips configured to deliver ultrasound energy to soft tissue. As noted above, each of the plurality of distal functional tips may be configured to provide a unique identifier when polled or otherwise interrogated by the ultrasound emitter 1502 and/or the ultrasound generator 1504, shown in FIG. 15. For example, the unique identifier may be associated with the type of the detected distal functional tip and/or with one or more procedures associated with the use of the detected type of distal functional tip. The detected distal functional tip may then be polled to obtain the unique identifier thereof, as shown at Block B172 of FIG. 17. According to Block B173, the signals to be applied to the ultrasonic soft tissue treatment device may then be configured based at least upon the obtained unique identifier. For example, such configuration of the signal to be applied to the ultrasonic soft tissue treatment device may comprise changing one or more of amplitude, frequency, power and on/off duty cycle of the signals to be applied to the ultrasound emitter assembly. As called for, at Block B174, the ultrasonic soft tissue treatment device may then be energized to cause the configured signals to be delivered to the ultrasonic soft tissue treatment device, transduced into ultrasound energy and delivered to the distal functional tip.

The method of FIG. 17 may further comprise inserting the distal functional tip into the soft tissue to apply ultrasonic energy thereto and energizing a motor (such as shown at reference numeral 502 in FIGS. 5A and 5B) that causes the distal functional tip to repetitively move within the soft tissue. The method may further comprise determining a number of uses the detected distal functional tip has undergone, based upon the obtained unique identifier. Using the unique identifier obtained from polling the distal functional tip, use of the detected distal functional tip may be enabled if the determined number of uses of the distal functional tip is less than a predetermined threshold and use thereof may be disallow if the determined number of uses is greater or equal to the predetermined threshold.

While certain embodiments of the inventions have been described, these embodiments have been presented by way of example only, and are not intended to limit the scope of the inventions. Indeed, the novel methods, devices and systems described herein may be embodied in a variety of other forms. Furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions. For example, those skilled in the art will appreciate that in various embodiments, the actual structures may differ from those shown in the figures. Depending on the embodiment, certain of the steps described in the example above may be removed, others may be added. Also, the features and attributes of the specific embodiments disclosed above may be combined in different ways to form additional embodiments, all of which fall within the scope of the present disclosure. Although the present disclosure provides certain preferred embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

1-45. (canceled)

46. A device, comprising:
an ultrasonic emitter, the ultrasound emitter being configured to be coupled to an ultrasound generator;
a waveguide assembly, the waveguide assembly comprising a proximal end coupled to the ultrasonic emitter and a distal end comprising a distal functional tip, wherein the ultrasonic emitter and waveguide assembly are integrated into a single, non-detachable assembly; and
a handle, the handle comprising an external surface and an internal surface that defines an interior cavity that is configured to at least partially encapsulate the ultrasonic emitter.

47. The device of claim 46, wherein the handle comprises a first half and a second half, wherein the first and second halves are configured to couple to one another.

48. The device of claim 46, further comprising a hermetic collar configured to provide a fluid-tight seal between the handle and at least a portion of the waveguide assembly.

49. The device of claim 46, further comprising a compressible material disposed within the internal cavity, the compressible material being configured to support the ultrasonic emitter within the internal cavity.

50. The device of claim 49, wherein the compressible material comprises acoustically isolating material.

51. The device of claim 49, wherein the compressible material comprises at least one annular support configured to surround the ultrasound emitter within the internal cavity of the handle.

52. The device of claim 49, wherein the compressible material comprises a ball defining a through bore through which the ultrasound emitter is disposed.

53. The device of claim 49, wherein the compressible material is configured to isolate the ultrasound emitter from the internal surface of the handle.

54. The device of claim 46, further comprising a movement generating mechanism that is configured to cause a repetitive movement of the distal functional tip of the waveguide assembly.

55. The device of claim 54, wherein the movement generating mechanism comprises an electric motor assembly, the electric motor assembly being coupled to the compressible material supporting the ultrasonic emitter within the internal cavity.

56. The device of claim 55, wherein the electric motor assembly comprises an eccentric motor.

57. The device of claim 46, wherein the handle generally defines a pistol grip shape.

58. The device of claim 46, further comprising a trigger coupled to the handle, the trigger being configured to cause the ultrasound emitter to move within the handle when depressed.

59. The device of claim 46, wherein the exterior surface comprises a porous layer.

60. The device of claim 46, wherein the waveguide assembly comprises an integrated concentrator, waveguide and distal function tip.

61. The device of claim 60, wherein the concentrator defines a conical shape.

62. The device of claim 60, wherein the concentrator comprises a surface having a shape of a Gaussian curve.

63-65. (canceled)

66. A device, comprising:
an integrated, one piece ultrasonic emitter and waveguide assembly, wherein the ultrasonic emitter is configured to be coupled to an ultrasound generator and deliver ultrasonic energy to a distal functional tip of the waveguide assembly;
a handle defining an interior cavity that is configured to receive the ultrasonic emitter; and
a hermetic collar configured to provide a fluid-tight seal between the handle and at least a portion of the waveguide assembly.

67. The device of claim 66, wherein the handle comprises a first half and a second half, wherein the first and second halves are configured to couple to one another.

68. The device of claim 66, further comprising a compressible material disposed within the internal cavity, the compressible material being configured to support the ultrasonic emitter within the internal cavity.
